(19) United States
(12) Patent Application Publication
(10) Pub. No.: US 2009/0325257 A1
(43) Pub. Date: Dec. 31, 2009

(54) Method for Influencing Living Cells Through Cell-Surface Interaction

(76) Inventors: Juliane Issle, Reckaange (LU); Uwe Hartmann, St. Ingbert (DE)

Correspondence Address: LAW OFFICES OF JAMES E. WALTON, PLLC, 1169 N. BURLESON BLVD., SUITE 107-328, BURLESON, TX, 76028, US

(21) Appl. No.: 12/375,874

(22) PCT Filed: Aug. 2, 2007
(86) PCT No.: PCT/DE2007/001377

§ 371 Date: Feb. 25, 2009

(30) Foreign Application Priority Data
Aug. 2, 2006 (DE) 10 2006 036 380.9

(51) Int. Cl. C12N 13/00

(52) U.S. Cl. 435/173.4

(57) ABSTRACT

The invention relates to a method for influencing living cells through cell-surface interaction where, for the cell-surface interaction, bioactive material is applied to magnetic carrier material, then this magnetic carrier material is applied to a magnetic carrier substrate, and this carrier substrate is combined with the living cells. The magnetic domain arrangement in the substrate can be altered by means of an external magnetic field in such a way that the structure formed by the magnetic carrier material is likewise changed. An in vitro alteration of the structure of the substrate is thus possible.

The invention is directed to a method for influencing living cells via cell-surface interaction in accordance with the preamble of claim 1.

It is known to the inventors that immobilization has already been used for the application of growth factors by means of simple adsorption on biocompatible surfaces. For adsorption it is necessary in advance to solve the growth factors in a solvent and to deposit the solution on a substrate and to dry it afterwards.

It is known as well in the state of the art to localize magnetic carrier material, so called “nano beads” on a substrate via magnetic force interaction, whereby bioactive material is bound to the surface of the nano beads and whereby the carrier material forms a surface structure on the substrate. For this purpose either magnetic substrates are used or in case of using non-magnetic substrates, a magnetic field is generated by providing magnets in the area of the substrate. A disadvantage with regard to this state of the art is that the surface structure formed by the magnetic carrier material causes interactions with the studied cell material, but a selective alternation or influence of the surface structure and hence of the cell-surface interaction is not possible at all or is at least very limited. Particularly it is not possible to systematically alter the geometry of the surface structure resulting from the spatial arrangement of the nano beads (for example circular, triangular, or labyrinthine arrangements).

The technical problem of the invention is to find a remedy and to influence living cells by cell-surface interactions by a systematic arrangement of the surface structure and to be able to carry out a time-dependent analysis of the structural changes.

This technical problem is solved by the present invention in accordance with claim 1, whereby the surface structure formed by the carrier material on the substrate can be systematically influenced by applying an external magnetic field.

Magnetic particles featuring diameters of less than 1 micron can be utilized as magnetic carrier material. These so called nano beads can for example be composed of the magnetic material magnetite. Such particles are commercially available with different reactive surface groups (carboxyl-, amino groups, etc.). Bioactive material, as for instance growth factors or proteins for various cell types, can be covalently bound to these surface groups.

Due to the covalent binding, the bioactive material is well bound to the carrier material. The magnetic carrier material is deposited on the magnetic substrate by magnetic interaction. By binding the bioactive material to the magnetic carrier material, the bioactive material follows the (electro)-magnetic structure of the carrier substrate and is distributed in the respective spatial structure on the magnetic carrier substrate.

In the state of the art, in case of immobilization, the bioactive material, as for example the growth factors, is on the one hand not in fluid environment anymore. Thereby the functionality may be influenced. On the other hand the biological material is not bound very strong to the substrate. This may lead to a detachment. Furthermore, the preparation of the state of the art does not allow for structuring in the nanometer to micrometer range. But in contrast to the latter this is possible with the present method which delivers insight into cell reactions on different structures.

In particular the present method allows for differentiation of stem cells.

In the present invention magnetic carrier substrates (thin layers) can be used which feature a certain domain structure. This domain structure can be influenced by external magnetic fields regarding shape and magnitude, whereby a high variability of the system is obtained.

One embodiment of the invention consists in generating the external magnetic field by a permanent magnet or electromagnet being positioned spatially variable.

It is also in the scope of the invention to provide electric conductive paths in the magnetic carrier substrate and to generate or change the external magnetic field by a current in the electric conductive paths.

It is possible as well to combine both manners for generating an external magnetic field.

If electric conductive paths are provided in the carrier substrate, the electromagnetic structure of the carrier substrate can by altered by applying an electric current flow in the conductive paths. The magnetic particles can follow these changes. In particular, when providing electric conductive paths, the possibility arises to generate a magnetic field of a determined structure in a determined and well adjustable way and simple manner, such that an external magnetic field may be superimposed to the magnetic field of the carrier substrate which is generated by a current flow in the electric conductive paths.

An embodiment of the invention consists in locally heating the carrier substrate by applying the external alternating magnetic field at least in this local area with a frequency in the kHz to MHz range.

This local heating can be performed for example with a local resolution in the range of some microns. The temporally fast changing magnetic fields can be generated on the one hand by magnets provided in the surrounding of the carrier substrate or on the other hand in an embodiment including electric conductive paths by currents featuring alternating currents in part. A movement of the magnetically bound particles can be induced by these magnetic fields resulting in heat dissipation. If this excitation is performed in certain areas of the substrate in a controlled manner, a local heating of the substrate and of the cells thereon can be achieved. This heating can be specifically used for a specific change or separation of the bio molecules which are coupled to the particles.

Thereby additional analysis of the specific influence of biological material may be performed. In contrast to other methods for local heating of tissue, this embodiment features the advantage of being able to spatially vary the magnetic fields very exactly and that it becomes possible to activate and influence certain cells in a defined manner, for analyzing these cells with respect to temperature influences. It is possible as well to generate a temperature gradient in the micrometer range.

In accordance with the invention an alternation of the time-dependent external magnetic field is provided.

At last it is possible as well to vary the magnetic field spatially.

Thereby the spatial arrangement of the magnetic carrier material can be advantageously influenced on the magnetic carrier substrate.

First it is possible to adjust the magnetization (i.e. the magnetic structure) of the carrier substrate before applying the magnetic carrier material.

Furthermore it is possible as well to apply a change to the magnetization after deposition of the magnetic carrier material. This can especially applied as well, when the biologically active material is already interacting with the living cells. The change of the magnetic structure of the carrier substrate und thus of the surface structure of the carrier material enables an in-vitro influence of the cell-surface interaction in this case. With that it is not only possible to obtain a spatial variation of the arrangement of the magnetic carrier material and hence of the biologically active material, but also to obtain a temporal variation of this arrangement.

The external magnetic field can be present independently and externally with regard to the carrier substrate. For generation of the magnetic field, permanent magnets or electromagnets in the surrounding of the carrier substrate may be used which may be changed spatially in their position such that either their field magnitude or the geometry of the magnetic fields can by varied with respect to the carrier substrate. Nevertheless, it is possible as mentioned before in connection with claim 1, to generate the external magnetic field by electric conductive paths embedded in the carrier substrate.

The external magnetic field can be temporally varied with frequencies in the mHz to MHz range. The geometry of the magnetic fields is provided in a variable and flexible way.

In general it has to be taken into account that the functionality of the bioactive material is not influenced.

By using the chip technology in which conductive paths are embedded in a biocompatible magnetic substrate, additionally the possibility for creation of carrier substrates with variable magnetic structures which can be influenced by the application of electric current, arises. Thereby the already mentioned external magnetic fields may be generated by current flows in the electric conductive paths.

In summary, the present invention renders the possibility of binding growth factors and special proteins, which are necessary for controlled influence of cells, modularly on a surface. With the arrangement it is in particular possible to classify influences which are caused by surface structures or certain arrangements of growth factors.

By the possibility of being able to structurally influence the described system of bio molecules and carrier substrates by external electromagnetic fields during the cell cultivation, new application areas with respect to the analysis of the reactions of biological systems, based on temporally and structurally variable carrier substrates, arise.

The invention further utilizes the occurring covalent binding of the bioactive material, as for example growth factors on the magnetic carrier material (nano beads). This is in any case a stronger and therewith more stable immobilization of the bio molecules than appearing in case of physisorption.

The resulting bond of the nano beads by magnetic forces on the carrier substrate is as well stronger than pure physisorption.

Furthermore, a reconfiguration of the surface structure by using weal<magnetic fields which have no influence on the cells is possible at every point in time, for example also during the application in cell culture.

Depending on the concentration and the size of the utilized functionalized nano beads and on the external magnetic fields, it is possible to provide the cells with different structures of growth factors in the micrometer and nanometer range.

Additionally, all preparation steps are applicable in liquid environment, such that it is assured that the bio molecules remain in physiological environment and do not have to be dried, as it is the case in the state of the art.

In particular in-vitro structural properties of the used substrate may be advantageously influenced with the present invention (time-space-profile). Growth factors, relevant bio molecules or proteins can be bound to certain areas of the carrier substrate in determinable concentrations.

A variable system for further cell research in general and research of stem cell differentiation is herewith provided which enables the analysis of structural influences on cellular mechanisms. Although the structures are variable, a stable immobilization of the bio molecules on the surface is guaranteed.

The invention is explained in further details by the attached figures. These figures show:

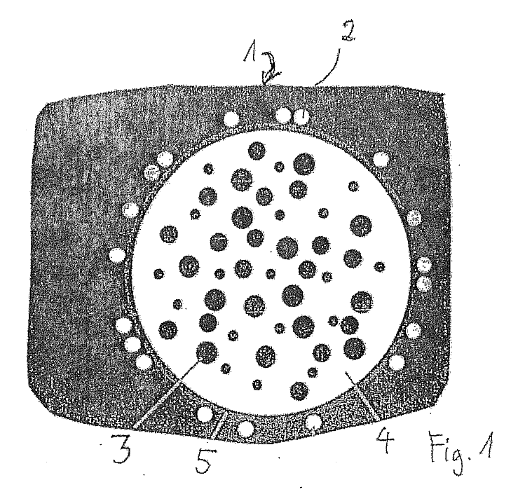

FIG. 1: a magnetic carrier material with attached bioactive material,

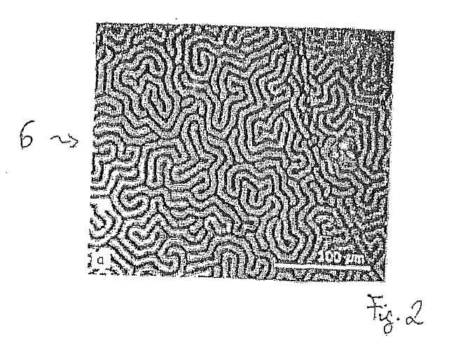

FIG. 2: a magnetic carrier substrate showing a typical distribution of magnetic domains,

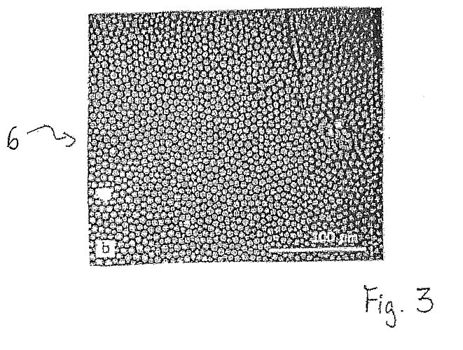

FIG. 3: the carrier substrate of FIG. 2 with a domain distribution altered by the method in accordance with the invention,

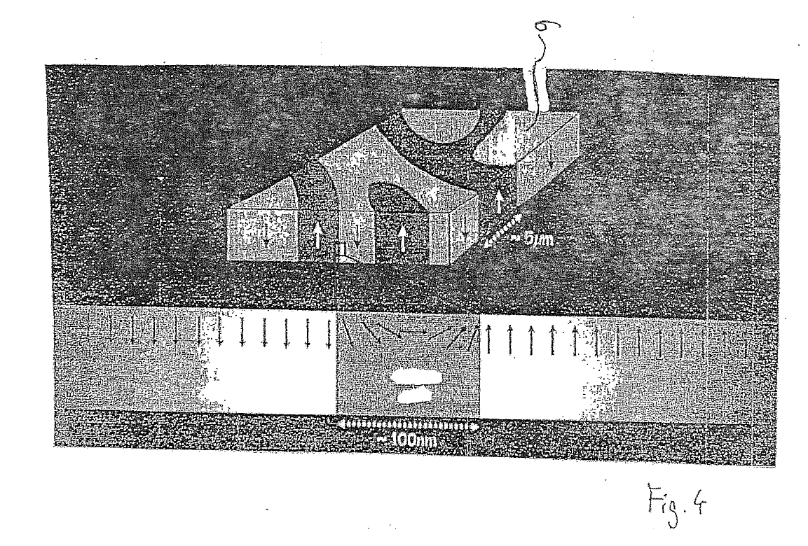

FIG. 4: a principle of the magnetic structure in the carrier substrate,

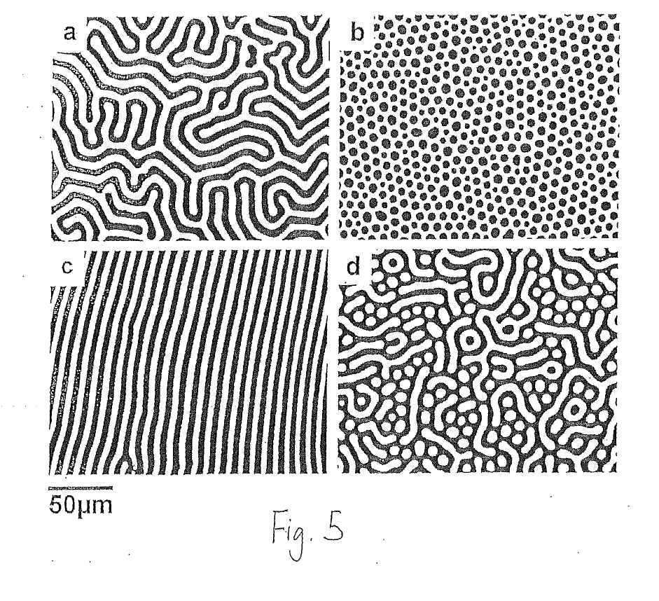

FIG. 5 an embodiment for the alternation of different magnetic domain structures,

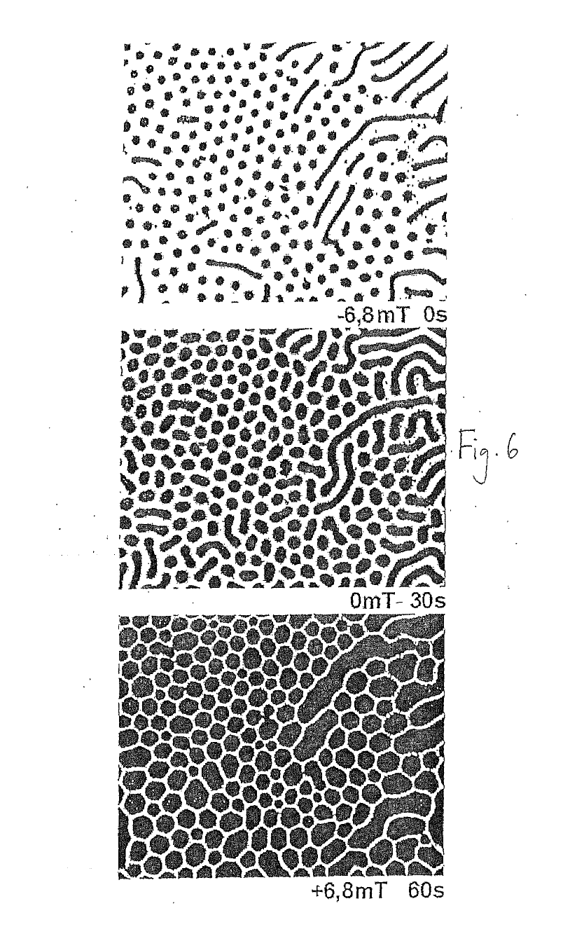

FIG. 6 an embodiment regarding the temporal variation of an already altered domain structure.

FIG. 1 shows a magnetic carrier material 1 with attached bioactive material 2. Carrier material 1 may consist of so called nano beads. These may consist for example of magnetite (Fe₃O₄) 3. The nano bead may have a total radius in the range of 100 nm to 500 nm. The single magnetite particles 3 feature a diameter of 20 nm, the nano bead consists of ca. 80% magnetite. This magnetite may be embedded in a matrix 4, which constitutes the remaining 20 vol. % and consists of a polysaccharide. The surface 5 of the nano bead can be composed of reactive molecules or of proteins which can be for instance COOH, NH₂ or other molecules or proteins.

By these molecules and proteins the magnetic carrier substrate 1 is doped with the bioactive material 2 which is bound to the surface of the nano beads by a covalent binding to the molecules or proteins.

FIG. 2 shows a magnetic carrier substrate 6 (here YIG, Yttrium Iron Garnet) with a magnetic domain distribution in accordance with the typical basic or delivery state as delivered by the producer. In this case the white, respectively the black areas, constitute domains with anti-parallel magnetization which is perpendicularly aligned to the plain of the substrate.

FIG. 3 shows the carrier substrate 6 after a temporally limited application and shutdown of an external magnetic field. This magnetic field may be generated by permanent magnets which are positioned appropriately or by electric coils to which a controllable current flow is applied.

It is possible as well to provide electric conducting paths on the carrier substrate 6, such that a magnetic field is generated when an electric current flow is applied to the electric conductive paths.

The magnetic or the magnetizable carrier substrate 6 can consist of the elements Y, Sm, Bi, Ga, Fe. These Garnet films are just an exemplary listing. In general all magnetic surfaces are possible as carrier substrate.

FIG. 4 shows a principle of the magnetization of the carrier substrate 6.

It can be seen, that areas are formed, which feature different orientations of the magnetic fields in accordance with the respective shown arrows. The orientations of the transition regions of carrier substrate 6 can be seen particularly in the magnification.

It has turned out that the nano beads accumulate exactly at these transition areas.

After such a carrier substrate 6 has been charged, it can be brought in contact with living cells. Therefore the living cells can be inserted in an aqueous solution in which the carrier substrate 6 is immerged.

In the following the invention is explained in more detail on the basis of two embodiments:

### Embodiment 1

The meander like domain distribution shown in FIG. 5a can be reconfigured by application of a temporally limited external magnetic field.

FIGS. 5b to 5d show exemplary domain distributions which can be achieved by applying different external magnetic fields in the same substrate as shown in FIG. 5a.

For this purpose for example a coil can be used which can be adjusted in its position angular-dependent of the magnetization of the substrate.

For arriving at the state of circular domain distribution in accordance with FIG. 5b, starting at the state shown in FIG. 5a, the coil is adjusted such that its magnetic field features an angle of 88° to 89° with respect to the magnetization of the substrate. Subsequently the substrate is brought into saturation by the magnetic field of the coil, establishing the new domain configuration after a slow shutdown of the field.

The stripe domains of FIG. 5c are established in an analog manner. But here an angle of about 70° between the magnetic field of the coil and the starting magnetization of the substrate is necessary.

The mixed state shown in FIG. 5d can be achieved by angular adjustments between the values mentioned in connection with the values of FIGS. 5b and 5c.

From any domain configuration one can return to the meander structure of FIG. 5a if the substrate is brought into saturation by the coil in parallel with its magnetization and the field is turned off afterwards.

### Embodiment 2

Once a certain domain state is achieved (stripe domains, mixed state, etc.), a coil can be used to alter this configuration regarding the domain width and/or the adjustment of the domains. For this purpose the field of the coil is set up in parallel with the magnetization of the substrate.

The magnetic structure of the carrier substrate can now be changed time-dependently, depending on the direction of the magnetic field (and therewith in the direction and the strength of the current in the coil).

FIG. 6 shows a temporally alternation of the domain structure in a period of 60s, during which the magnetic field strength rose linearly from −6,8 mT to 6,8 mT. For maintaining a certain domain width the respective magnetic field strength has to be maintained.

As described before, the magnetic carrier material follows the structural changes of the substrate after being brought on the substrate and accumulates preferably in the transition area between the single domains. Thus, it features a temporally and spatially variable structure in cell culture for enabling analysis of time dependent cellular processes.

1-6. (canceled)

7. A method for influencing living cells by cell-surface interaction, comprising:
applying bioactive material to a magnetic carrier material;
depositing the magnetic carrier material on a magnetic carrier substrate, thereby forming a surface structure;
placing the magnetic carrier substrate into contact with living cells; and
applying an external magnetic field to the surface structure, so as to systematically influence the surface structure.

8. The method according to claim 7, wherein the external magnetic field is generated by a selectively positioned permanent magnet.

9. The method according to claim 7, wherein the external magnetic field is generated by a selectively positioned electromagnet.

10. The method according to claim 7, further comprising:
providing an electric conductive path in the magnetic carrier substrate, such that the external magnetic field is generated by a current flow in the electric conductive path.

11. The method according to claim 7, further comprising:
providing an electric conductive path in the magnetic carrier substrate, such that the external magnetic field is changed by a current flow in the electric conductive path.

12. The method according to claim 7, further comprising:
adjusting the magnetic field in the KHz to MHz range, thereby allowing the magnetic carrier material to be locally heated.

13. The method according to claim 7, further comprising:
time-dependently altering the external magnetic field.

14. The method according to claim 7, further comprising:
spatially altering the external magnetic field.
